(19) United States
(12) Patent Application Publication
(10) Pub. No.: US 2022/0340880 A1
(43) Pub. Date: Oct. 27, 2022

(54) A method for obtaining pluripotent stem cell-derived airway basal-like cells and an airway epithelium model

(71) Applicant: NEWCELLS BIOTECH LIMITED, Newcastle Upon Tyne (GB)

(72) Inventors: Ivo DJIDROVSKI, Newcastle upon Tyne (GB); Lyle ARMSTRONG, Newcastle upon Tyne (GB)

(21) Appl. No.: 17/763,888

(22) PCT Filed: Sep. 23, 2020
(86) PCT No.: PCT/GB2020/052310

§ 371 Date: Mar. 25, 2022

(30) Foreign Application Priority Data
Sep. 26, 2019 (GB) 1913923.7

(51) Int. Cl. C12N 5/071; C12N 5/074

(52) U.S. Cl. CPC C12N 5/0689; C12N 5/0696; C12N 2500/90; C12N 2501/155

(57) ABSTRACT

The present invention relates to methods for obtaining a substantially pure population of pluripotent stem cell-derived airway basal-like cells. It also relates to a method of obtaining an in vitro pluripotent stem cell-derived airway epithelium model, utilising the pluripotent stem cell-derived airway basal-like cells. The invention further relates to an in vitro airway epithelial model, or lung model, which can be used for disease modelling and/or drug screening and in particular to an in vitro model for SARS-CoV-2 infection and for screening for agents effective against infection with SARS-CoV-2 i.e. COVID-19 and methods of using the same.

### TECHNICAL FIELD

The present invention relates to methods for obtaining a substantially pure population of pluripotent stem cell-derived airway basal-like cells. It also relates to a method of obtaining an in vitro pluripotent stem cell-derived airway epithelium model, utilising the pluripotent stem cell-derived airway basal-like cells.

The invention further relates to an in vitro airway epithelial model, or lung model, which can be used for disease modelling and/or drug screening and in particular to an in vitro model for SARS-CoV-2 infection and for screening for agents effective against infection with SARS-CoV-2 i.e. COVID-19 and methods of using the same.

### BACKGROUND

According to NHS England, respiratory disease affects one in five people and is the third biggest cause of death in England. Recently, increased rates of certain respiratory diseases such as asthma and chronic obstructive pulmonary disease (COPD) has been attributed to increased exposure to inhaled toxic particles.

The human respiratory system primarily comprises the lungs and the airway. The human airway begins at the openings of the nasal cavity and mouth, air enters these openings and moves towards the lungs through the trachea. The trachea branches to form two bronchi which carry air into the lungs, the bronchi further branch to form bronchioles which terminate in small sacs known as alveoli which facilitate gas exchange with the circulatory system.

Toxic particles and pathogens inhaled through the nasal cavity and mouth can be filtered out by the airway before reaching the alveoli. The human airway comprises intrinsic defences against pathogens and toxic particles, the mucus producing cells of the airway epithelium excrete a layer of mucus which lines the airway and traps harmful molecules.

Beating ciliated cells line the airway epithelium and assist with both spreading the mucus layer throughout the airway and expelling harmful molecules which have become trapped in the mucus layer by moving them to the mouth to be coughed or swallowed.

In order to study respiratory diseases and the effects of inhalation toxicity on the development and progression of respiratory diseases, access to populations of airway cells and reliable, consistent models that closely replicate the in vivo tissue is important. Multiple different in vivo and in vitro models have been developed for studying respiratory diseases and inhalation toxicity. Commonly, rodents such as rats and mice have been used as in vivo models to study respiratory diseases and to assess inhalation toxicity. The physiological, genetic, and structural differences between the human and rodent airway often makes the translation of rodent studies to humans difficult and unpredictable. Increasingly, ethical concerns and high costs associated with animal studies have driven research towards the development of in vitro alternatives to rodent models.

Many of the in vitro models for assessing inhalation toxicity and studying respiratory diseases have been based on monolayer cultures of immortalised human lung cells. While these models are easily accessible and cost efficient, they do not replicate the layered structure of the human airway epithelium. Moreover, these models do not allow for personalised disease modelling and examining the physiological effects of different patient mutations.

The development of in vitro models having a closer structural resemblance to the human airway epithelium has focussed on the use of air-liquid interface (ALI) culture. Culturing cells on ALI has resulted in the production of in vitro models containing differentiated, functional airway epithelium cells which are able to organise into a pseudostratified structure, as seen in the human airway epithelium. However, this approach has only been successfully achieved by culturing primary human bronchial epithelial cells on ALI.

Use of primary cells in the creation of in vitro airway epithelium models presents major disadvantages, firstly the availability of primary cells is extremely limited by the lack of availability of suitable donor material. In addition, the primary cells cannot be kept indefinitely in culture, further limiting the availability of primary cells that can be used as starting material. Furthermore, genetic, and epigenetic differences between individual donors leads to differences in the resulting models. These differences can change the way that each model responds in experiments, making comparisons between data derived from individual models extremely difficult and unreliable.

An alternative in vitro model has been described by Konishi et al., (2016), which uses human induced pluripotent stem cells (hiPSCs) to derive an in vitro human airway epithelium spheroid model. Use of iPSCs as a starting material overcomes many of the problems with the previously described models as these cells are much more readily available than primary cells and can be expanded readily. iPSCs can also be derived from different individuals with or without genetic conditions, or genetically modified, to create different disease models and can be differentiated into almost any cell type given the correct conditions. Despite the advantages associated with use of iPSCs, the model described by Konishi et al., (2016) does not replicate the pseudostratified structure of the human airway epithelium and moreover the differences between the individual spheroids makes it difficult to extrapolate meaningful data through use of this model.

Attempts have been made to isolate the airway progenitor cells, from the spheroids obtained by the method of Konishi et al., (2016) and to use these cells to obtain a pseudostratified airway epithelium model by culturing on ALI. However, the airway epithelium spheroids contain an insufficient quantity of basal-like cells for isolation and further culture on ALI.

### COVID 19

The outbreak of the novel coronavirus disease, COVID-19, caused by coronavirus SARS-CoV-2 has been designated as a pandemic by the World Health Organisation and is currently a significant threat to human health. As of June 2020, there were 7.7 million confirmed cases of whom 427,400 have died. The pandemic poses major challenges to global healthcare systems and could have severe consequences for the global economy if the spread of the virus is not effectively controlled.

The causative agent of COVID-19, SARS-CoV-2 has been shown to attack the respiratory system resulting in viral pneumonia but it may also affect the gastrointestinal system, heart, kidney, liver, and central nervous system leading to multiple organ failure. Previous studies have shown that SARS-CoV predominantly infects airway and alveolar epithelial cells, vascular endothelial cells, and macrophages using the angiotensin converting enzyme receptor (ACE2) for entry. Rapid viral replication in these cells can lead to massive epithelial and endothelial cell apoptosis causing the release of pro-inflammatory cytokines which can potentially cause lung damage and diminished patient survival. This is exemplified by the observation that in SARS-CoV-2 infected individuals, interleukin (IL)-6, IL-10 and TNFα surges during illness and declines during recovery. Severely affected patients who require intensive care treatment can be distinguished by significantly higher levels of IL-6, IL-10 and TNFα and fewer CD4+ and CD8+ T cells. Although it is likely that ingress of large numbers of cytokine secreting inflammatory macrophages into the lung tissue accounts for a significant proportion of the cytokines detected in such cases, the initial damage to the lung epithelial and endothelial cells probably contributes not only to the overall concentration of cytokines, but may also be responsible for recruitment of inflammatory macrophages. Other mechanisms besides apoptosis can lead to activation of the inflammasome. The binding of SARS-CoV-2 to the Toll like receptor causes release of pro-IL-1β which is cleaved by caspase-1, followed by inflammasome activation and production of active mature IL-1β which is a mediator of lung inflammation, fever and fibrosis. To underline this, suppression of pro-inflammatory IL-1 family members and IL-6 have been shown to have a therapeutic effect in many inflammatory diseases, including viral infections. IL-1β can also enhance the constitutive detachment (or shedding) of an enzymatically active ectodomain fragment of ACE2 from the airway epithelial cells, an event associated with acute lung injury [10, 11]. Interestingly, binding of SARS-CoV-2 infection is associated with ACE2 downregulation and ectodomain shedding thought to be induced by the SARS-CoV S protein. How the released form of ACE2 (the so-called soluble or sACE2) causes lung damage is not completely clear but it seems to be tightly coupled to TNFα production, so it may be involved in inflammatory response to SARS-CoV-2 infection.

Despite this understanding of the mechanisms by which SARS-CoV type viruses damage the lung epithelia and vascular endothelia, as of September 2020 there are no effective treatments for the resulting COVID-19 disease. Current management of COVID-19 is supportive, and respiratory failure from acute respiratory distress syndrome (ARDS) is the leading cause of mortality. In view of this, there is an urgent and currently unmet need for model systems that can function as high throughput pre-clinical tools for the development of novel, effective therapies for COVID-19

The present invention aims to obviate or mitigate the problems associated with the prior art by providing a substantially pure population of pluripotent stem cell-derived airway basal-like cells and a method of producing the same. Also provided is a method of obtaining an in vitro pluripotent stem cell-derived airway epithelium model using said substantially pure population of pluripotent stem cell-derived airway basal-like cells. Further provided is an in vitro model system that can function as high throughput pre-clinical tool for the development of novel, effective therapies for COVID-19.

### SUMMARY OF THE INVENTION

According to a first aspect of the present invention there is provided a method for obtaining a substantially pure population of pluripotent, stem cell-derived, airway basal-like cells comprising the steps: differentiating a population of pluripotent stem cells to obtain a heterogeneous population of pluripotent stem cell-derived lung progenitor cells; culturing the pluripotent stem cell-derived lung progenitor cells in the presence of feeder cells and a rho-kinase inhibitor to obtain a population of pluripotent stem cell-derived airway basal-like cells; and culturing the pluripotent stem cell-derived lung progenitor cells and feeder cells in a serum-free medium to obtain a substantially pure population of pluripotent stem cell-derived airway basal-like cells.

Advantageously, culturing the pluripotent stem cell-derived lung progenitor cells and feeder cells together in a serum-free medium results in the death of the feeder cells, leaving a substantially pure population of pluripotent stem cell-derived airway basal-like cells without having the need to sort the cells.

Preferably, the pluripotent stem cells are differentiated for a period of between 10 and 20 days. More preferably, the pluripotent stem cells are differentiated for a period of between 12 to 16 days. Most preferably, the pluripotent stem cells are differentiated for a period of 14 days.

Preferably, the pluripotent stem cells are differentiated for a first time period in the presence of activin A and a GSK3β inhibitor; for a second time period in the presence of a BMP inhibitor and a TGFβ inhibitor; and for a third time period in the presence of BMP4, retinoic acid and a GSK3β inhibitor.

Preferably, the first time period is between 1 and 12 days; the second time period between 2 and 8 days; and the third time period between 2 and 8 days. More preferably, the first time period is 5 days; the second time period is 4 days; and the third time period is 4 days.

Preferably, the pluripotent stem cell-derived lung progenitor cells are cultured as a monolayer on the feeder cells in the presence of a rho-kinase inhibitor.

Preferably, the pluripotent stem cell-derived lung progenitor cells are cultured as a monolayer on the feeder cells in the presence of a rho-kinase inhibitor with a serum-free medium for a period of between 3 and 10 days. More preferably, the pluripotent stem cell-derived lung progenitor cells are cultured as a monolayer on the feeder cells in the presence of a rho-kinase inhibitor with a serum-free medium for a period of 7 days. Preferably, the pluripotent stem cells are induced pluripotent stem cells (iPSCs).

Most preferably, the induced pluripotent stem cells (iPSCs) are mammalian induced pluripotent stem cells, more preferably human induced pluripotent stem cells (hiPSCs).

Advantageously, the use of hiPSCs allows collection of cells from the patient to be treated without the need for an invasive biopsy procedure. Pluripotent stem cell-derived airway basal-like cells derived from hiPSCs can be used in regenerative medicine applications and transplantation without risk of rejection.

Optionally, the induced pluripotent stem cells are derived from a patient without any known genetic disorder or respiratory disease.

Optionally, the induced pluripotent stem cells are derived from a patient with a known genetic disorder or respiratory disease.

Advantageously, by using induced pluripotent stem cells derived from a patient with a known genetic disorder or respiratory disease, the pluripotent stem-cell derived airway basal-like cells can be used to produce a disease model.

Preferably, the substantially pure population of pluripotent stem-cell derived airway basal-like cells comprises cells expressing one or more airway basal cell markers.

More preferably, at least 70% of the substantially pure population of pluripotent stem-cell derived airway basal-like cells express one or more airway basal cell markers.

Most preferably, at least 90% of the substantially pure population of pluripotent stem-cell derived airway basal-like cells express one or more airway basal cell markers.

Optionally, the airway basal-cell markers are ΔNP63, NGFR, cytokeratin 14 and integrin α6.

Preferably, the substantially pure population of pluripotent stem-cell derived airway basal-like cells contains cells having a cuboidal morphology.

Preferably, the substantially pure population of pluripotent stem-cell derived airway basal-like cells contains cells having enlarged nuclei.

Preferably, the pluripotent stem cell-derived lung progenitor cells are plated at a 1:1 ratio with the feeder cells.

Preferably, the feeder cells are mouse fibroblast cells.

Optionally, the feeder cells are 3T3-J2 cells. These may be preferred in some cases.

Preferably, the feeder cells secrete leukaemia inhibitory factor (LIF).

Preferably, the feeder cells are mitotically inactivated.

Optionally, the feeder cells are mitotically inactivated by irradiation.

Optionally, the feeder cells and pluripotent stem cell-derived lung progenitor cells are cultured in a serum-free medium.

Optionally, the feeder cells and pluripotent stem cell-derived lung progenitor cells are cultured in a media supplemented with LIF.

Preferably, the rho-kinase inhibitor is used at a concentration of between 5 μM and 30 μM. Most preferably, the rho-kinase inhibitor is used at a concentration of around 10 μM.

According to another aspect of the present invention there is provided a method of treating an individual having respiratory disease, comprising implanting one or more pluripotent stem-cell derived airway basal-like cells as described above.

Another aspect of the present invention relates to use of the substantially pure population of pluripotent stem-cell derived airway basal-like cells in a drug discovery screen; toxicity assay; inhalation assay; research of differentiation pathways; research of disease aetiology.

According to another aspect of the present invention, there is provided a substantially pure population of pluripotent stem cell-derived airway basal-like cells wherein at least 50% of the cells express NGFR and at least 70% of the cells express Integrin α6; preferably at least 60% of the cells express NGFR and at least 80% of the cells express Integrin α6; more preferably at least 70% of the cells express NGFR and at least 90% of the cells express Integrin α6 and optionally, at least 50%, more preferably 70%, of the cells express cytokeratin 14.

According to another aspect of the present invention, there is provided a substantially pure population of pluripotent stem cell-derived airway basal-like cells wherein at least 50% of the cells express NGFR, at least 70% of the cells express Cytokeratin 14 and at least 70% of the cells express Integrin α6; preferably at least 60% of the cells express NGFR, at least 80% of the cells express Cytokeratin 14 and at least 80% of the cells express Integrin α6; more preferably at least 70% of the cells express NGFR, at least 95% of the cells express Cytokeratin 14 and at least 90% of the cells express Integrin α6.

Preferably, the pure population of induced pluripotent stem cell-derived airway basal-like cells have a cuboidal morphology and/or enlarged nuclei.

According to another aspect of the present invention, there is provided a method for obtaining an in vitro pluripotent stem cell-derived airway epithelium model comprising the steps of: obtaining a population of pluripotent stem cell-derived airway basal-like cells as described above; and culturing the population of pluripotent stem cell-derived airway basal-like cells on an air-liquid interface to obtain an in vitro pluripotent stem cell-derived airway epithelium model.

As the methods produce a large supply of cells with the genetic background of the donor this can produce a standardised, reproducible model of an airway or lung that allows the effects of large numbers of agents to be screened. However, it could also be used to produce a personalised model of a single individuals airway to allow for more personalised testing and the identification of more personalised treatments and dosage amounts.

Advantageously, the in vitro pluripotent stem cell-derived airway epithelium model provides a cost-effective model which resembles the naturally occurring airway epithelium.

Preferably, the in vitro pluripotent stem cell-derived airway epithelium model comprises cells expressing one or more airway epithelial cell markers.

Optionally, the airway epithelial cell markers are; Club Cell Protein 10, Mucin 1, ΔNP63 and Acetylated Tubulin.

Preferably, the in vitro pluripotent stem cell-derived airway epithelium model has a substantially layered structure which resembles a naturally occurring airway epithelium and comprises a plurality of cell types selected from basal cells, ciliated cells, goblet cells and club cells.

Advantageously the layered structure of the in vitro pluripotent stem cell-derived airway epithelium model resembles a naturally occurring airway epithelium.

Optionally, the air-liquid interface is provided by culturing the cells on an insert placed in a cell culture vessel.

Preferably, the pluripotent stem cell-derived airway basal-like cells are cultured on the top of the insert in the cell culture vessel and cell culture medium is added beneath the insert such that the cells on the top of the insert are exposed to the atmosphere.

Preferably, the air-liquid interface culture is allowed to mature for 5 or more days. More preferably, the air-liquid interface culture is allowed to mature for 14 or more days. Most preferably, the air-liquid interface culture is allowed to mature for 21 or more days.

Optionally, the in vitro pluripotent stem cell-derived airway epithelium model is used in a drug discovery screen; toxicity assay; inhalation assay; research of differentiation pathways; pharmacokinetic studies of a compound; pharmacodynamic studies of a compound; studies of disease aetiology.

According to another aspect of the present invention there is provided an in vitro pluripotent stem cell-derived airway epithelium or lung model which expresses one or more airway epithelial cell markers selected from Club Cell Protein 10, Mucin 1, ΔNP63 and Acetylated Tubulin; and which has a substantially layered structure which resembles a naturally occurring airway epithelium and comprises a plurality of cell types selected from basal cells, ciliated cells, goblet cells and club cells.

According to another aspect of the present invention there is provided a COVID 19 lung model, comprising an in vitro pluripotent stem cell-derived airway epithelium model described above, or which is obtained by the methods described above, which has been infected with coronavirus SARS-Cov-2.

Preferably, the COVID-19 lung model comprises cilia which have a cilia beat frequency of 11±1 Hz to 14±1 Hz, or a cilia beat frequency 12.4±1.6 Hz to 13.6±2.0 Hz, or a cilia beat frequency of 12.4±1.6 Hz, or a cilia beat frequency of 13.6±2.0 Hz.

Preferably, the COVID-19 lung model cells show increased secretion of IL-6, preferably increased secretion of IL-6 IL-10 and TNFα, after infection with coronavirus SARS-Cov-2 compared to a model where cells are not infected with coronavirus SARS-Cov-2.

Preferably IL-6 secretion equating to greater than 2-fold increase, more preferably a greater than 2.5 fold increase, most preferably a greater than 2.9 fold increase, and optionally a four-fold increase above that of the uninfected control 48 hours post infection.

According to another aspect of the present invention there is provided an ex-vivo or in vitro method of screening for prophylactic and therapeutic agents for COVID-19, comprising the steps of;

infecting an in vitro pluripotent stem cell-derived airway epithelium model obtained by the method described above with coronavirus SARS-CoV-2 to give an infected model;

bringing a test agent into contact with the infected model; and

detecting or measuring a response in the infected model.

Various further features and aspects of the invention are defined in the claims.

Unless defined otherwise, all technical and scientific terms used herein have the meaning commonly understood by a person skilled in the art to which this invention belongs.

To assist the reader, the following terms have the meanings ascribed to them below, unless specified otherwise.

The term “disease” refers to any condition or disorder that damages or interferes with the normal function of a cell, tissue, or organ. For example, chronic obstructive pulmonary disease (COPD), asthma and cystic fibrosis are diseases affecting the respiratory system.

The term “marker” refers to any protein or polynucleotide analyte having an expression level or activity associated with a particular cell type. In one embodiment, proteomics can be used to measure the levels of markers associated with cell differentiation.

The term “pluripotent stem cells (PSCs)”, also commonly known as PS cells, encompasses any cells that can differentiate into nearly all cells, i.e., cells derived from any of the three germ layers (germinal epithelium), including endoderm (interior stomach lining, gastrointestinal tract, the lungs), mesoderm (muscle, bone, blood, urogenital) and ectoderm (epidermal tissues and nervous system). PSCs can be the descendants of totipotent cells, derived from embryos (including embryonic germ cells) or obtained through induction of a non-pluripotent cell, such as an adult somatic cell, by forcing the expression of certain genes.

The term “induced pluripotent stem cells” also commonly abbreviated to iPS cells or iPSCs, refers to a type of pluripotent stem cell artificially derived from a normally non-pluripotent cell, such as an adult somatic cell, by inducing a “forced” expression of certain genes.

The term “basal cells” refers to the undifferentiated cells of the airway epithelium, having the capability to differentiate into any of the specialised cell types of the respiratory system such as ciliated cells, goblet cells and club cells and having the capability to generate more basal cells.

The term “basal-like cells” refers to cells sharing the characteristics of basal cells.

The term “medium”, also referred to as cell culture medium or culture medium, refers to a medium for culturing cells containing nutrients that maintain cell viability and support cell proliferation.

The term “serum-free medium” refers to a medium free of unadjusted or unpurified serum. The serum-free medium may be a commercially available medium such as Bronchial Epithelial Cell Growth Medium (BEGM) manufactured by Lonza®. The serum-free medium may contain a serum replacement, the serum replacement may be a commercially available serum replacement for example, Knockout Serum Replacement manufactured by Invitrogen®.

The terms “express”, “expressing” and “expression” refer to the cellular processes involved in producing RNA and proteins and as appropriate, secreting proteins, including where applicable, but not limited to, transcription, translation, folding, modification and processing. Expressed markers include RNA transcribed from a gene and polypeptides obtained by translation of mRNA transcribed from a gene.

The terms “treat”, “treating” and “treatment”, and the like refer to reducing or ameliorating a disorder and/or symptom(s) associated therewith. It will be appreciated that, although not precluded, treating a disorder, disease or condition does not require that the symptoms associated therewith be completely eliminated.

The term “rho-kinase inhibitor”, also known as ROCKi and RKI, refers to any compound which acts to inhibit the activity of rho-associated protein kinases. Examples of rho-kinase inhibitors include, but are not limited to, Y-27632, Y-30141, RKI-1447, Y-39983, AT877 and fasudil.

The term “feeder cells” refers to cells used to support the growth of another cell type. Feeder cells often growth-arrested cells which excrete growth factors into the medium which support the growth and proliferation of other cells. Examples of feeder cells include but are not limited to, HeLa cells, 3T3 cells, human dermal fibroblasts, murine embryonic fibroblast (MEF) cells, human amniocytes, human bone marrow stromal cells, human amniotic epithelial cells.

The term “pluripotent stem cell-derived lung progenitor cells” (also known as “ventral anterior foregut cells”) refers to pluripotent stem cells which have partially differentiated and are destined to differentiate into thyroid gland or lung in the presence of the appropriate growth factors. These cells are no longer capable of differentiating into mesoderm and ectoderm. And express markers such as nkx2.1 and foxa2.

### BRIEF DESCRIPTION OF THE DRAWINGS

Embodiments of the present invention will now be described by way of example only with reference to the accompanying drawings, where like parts are provided with corresponding reference numerals and in which:

FIG. 1 shows the results of flow cytometry analysis of the substantially pure population of pluripotent stem cell-derived airway basal-like cells;

FIG. 2a shows schematically the method of differentiation of iPSCs into a substantially pure population of airway basal-like cells; 2b and 2c show images of cells at time points throughout the method;

FIG. 3a shows schematically the method of obtaining an in vitro pluripotent stem cell-derived airway epithelium model or lung model from the substantially pure population of airway basal-like cells; 3b is a figurative indication of the air-liquid interface and the manner in which the cells arrange themselves in layers i.e. basal cells, intermediary cells, mucus producing cells, ciliated cells and produce a mucus layer on top; 3c shows images of cells at time points throughout the method—cells with beating cilia that were visible after 4 weeks of culture; 3d is a graph showing changes in Trans epithelial electrical resistance (TEER) values over time during the method; 3e is an image of a section of a model indicate the presence of a pseudostratified epithelium; 3f are images of the resulting pseudostratified epithelium; cells expressing the basal cell marker, p63 are enriched in a layer adjacent to the membrane of the transwell insert (sixth panel), the presence of club cell protein 10 is indicated by club cells (fourth panel), and expression of mucin-1 is indicative of goblet cells (fifth panel);

FIG. 4 is a table showing the beat frequency of cilia present on the iPSC derived model; and

FIG. 5a shows images of SARS-CoV-2 invasion into in vitro pluripotent stem cell-derived airway epithelium models; 5b is a table showing the number of viral particles present in the cells of the construct post infection; 5c shows graphs indicating secretion levels of inflammatory cytokines in response to SARS-CoV-2 infection (grey) compared to non-infection (black); and 5d shows images detecting the presence of S-protein by immunohistochemical location (i—uninfected control model, ii—secondary antibody only control and iii—IHC localisation of S-protein).

### DETAILED DESCRIPTION

In some embodiments, exemplary iPSCs include, but are not limited to, SBAD2 (AD2), SBAD3 (AD3) and SBAD4.

### Method for Obtaining a Substantially Pure Population of Pluripotent Stem Cell-Derived Airway Basal-Like Cells

Human induced pluripotent stem cell-derived airway basal-like cells were generated by the following methodology. The method below describes one example of how the pluripotent stem cell-derived airway basal-like cells could be obtained according to one embodiment of the present invention.
1. Day 1-6: Plate iPSCs on Matrigel-coated plates. Culture iPSCs to 90% confluency in definitive endoderm (DE) medium (advanced RPMI medium supplemented with 100 ng/ml human activin A, 50 U/ml penicillin/streptomycin, 1 μM CHIR, 10 μl/ml and B27 supplement). Incubate the cells at 37° C., 5% CO₂. Replace medium every day for 6 days.
2. Day 6-10: At day 6, replace the DE medium with anterior foregut endoderm (AFE) medium (DMEM/F12 supplemented with GlutaMax 1:100, 50 U/ml penicillin/streptomycin, 100 ng/ml human recombinant noggin and 10 μM SB431542). Incubate the cells at 37° C., 5% CO₂. Replace AFE medium with fresh AFE medium at day 8.
3. Day 10-14: At day 10, replace AFE medium with ventralisation of anterior foregut endoderm cells (VAFE) medium (DMEM/F12 medium supplemented with GlutaMax 1:100, penicillin/streptomycin, B27 supplement 1:50, 20 ng/ml human recombinant BMP4, 1 μM all-trans retinoic acid (ATRA) and 1 μM CHIR). Incubate the cells at 37° C., 5% CO₂. Replace the VAFE medium with fresh VAFE medium at day 12.
4. Day 14 onwards: Dissociate the cells with trypsin-EDTA, add stop medium (DMEM supplemented with 10% FBS) to cells to stop dissociation when appropriate. Centrifuge dissociated cells at 400×g for 5 minutes. Aspirate trypsin and re-suspend cells in basal cell medium (BEGM supplemented with 10 μM Rock inhibitor). Plate the cells at a 1:1 ratio onto irradiated T3T plates and incubate at 37° C., 5% CO₂. Replace the basal cell medium with fresh basal cell medium every 2 or 3 days until the cells reach 80% confluency. Cells can be passaged at a ratio of 1:6 onto freshly irradiated plates of T3T cells in basal cell medium. To passage cells, add trypsin for 1 minute, aspirate trypsin and add fresh trypsin for 4 minutes before adding stop medium.

It is preferred that ROCK inhibitor and serum free media are added to the cells substantially simultaneously, as above, however it is also possibly to resuspend cells in the serum free media and then later add the ROCK inhibitor.

### Characterisation of Substantially Pure Population of Pluripotent Stem Cell-Derived Airway Basal-Like Cells by Flow Cytometry

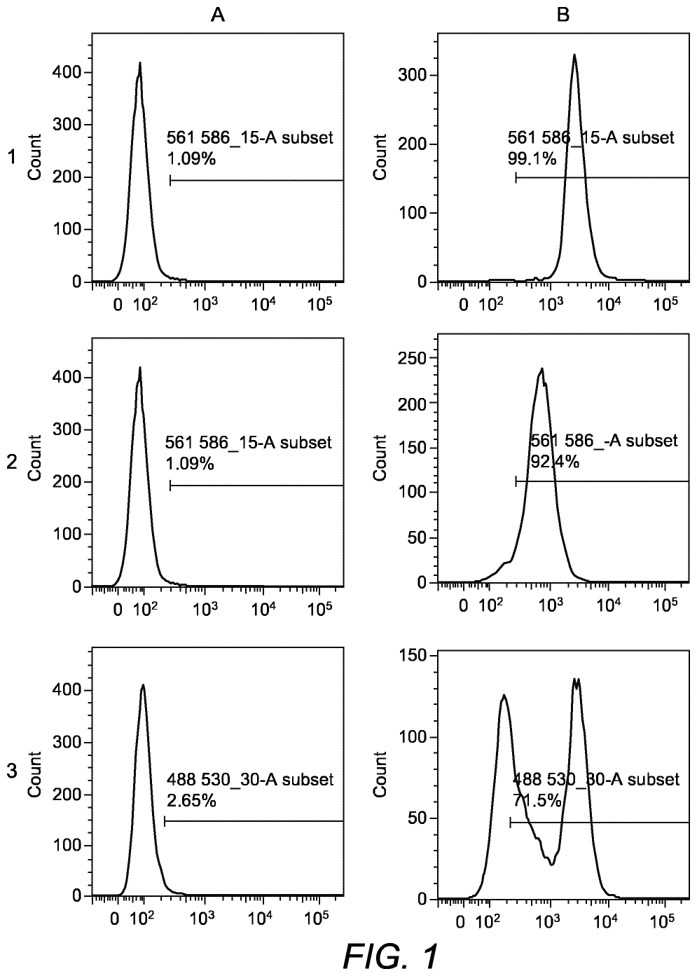

The characterisation of the purity of the population has been analysed by flow cytometry and the results are shown in FIG. 1. An exemplary method of carrying out flow cytometry is detailed below.
1. The substantially pure population of pluripotent stem cell-derived airway basal-like cells are obtained according to the above method and washed with ice cold PBS. The cells are centrifuged at 200×g for 4 minutes before adding trypsin and incubating at 37° C., 5% CO₂ for 5 minutes. PBS is then added to the cells to deactivate the trypsin.
2. Cells are centrifuged for 4 minutes at 200×g and subsequently fixed in 2% paraformaldehyde (PFA) for 10 minutes at 37° C. Cells are then washed three times in ice cold PBS and resuspended in FACS buffer (2% foetal bovine serum (FBS) in PBS).
3. Primary antibodies are added to the cells at an appropriate concentration (mouse anti-integrin α6 1:100; mouse anti-NGFR 1:100; mouse anti-cytokeratin 14 1:100) and incubated at room temperature for 30 minutes.
4. Cells are then washed three times with FACS buffer and incubated with an appropriate secondary antibody (rabbit anti-mouse Alexa 488 1:1000) for 30 minutes at room temperature. Cells are subsequently washed to remove the secondary antibody.
5. Samples are then analysed on an appropriate flow cytometer; in this example a Fortessa flow cytometer is used. Cell populations can be identified based on size (side scatter), granularity (forward scatter) and fluorescence. Suitable software, such as FlowJo software is used to analyse the data. A minimum of 10,000 cells should be analysed per sample.

FIG. 1 shows the results of flow cytometry analysis of the substantially pure population of pluripotent stem cell-derived airway basal-like cells. Basal-like cells were identified by the presence of three basal cell markers—integrin α6, cytokeratin 14 and NGFR. Column A shows a population of control cells and column B shows a substantially pure population of pluripotent stem cell-derived airway basal-like cells. Row A shows the cells positive for cytokeratin 14, row B shows the cells positive for integrin α6 and row C shows the cells positive for NGFR.

The results in FIG. 1, graphs 1A and 1B show that the percentage of cells positive for cytokeratin 14 in the control cell population is 1.09% and graph 1B shows the percentage of cells positive for cytokeratin 14 in the substantially pure population of pluripotent stem cell-derived airway basal-like cells is 99.1%.

The results in FIG. 1, graph 2A demonstrates that the percentage of control cells which are positive for integrin α6 in the control cell population is 1.09% and graph 2B demonstrates that the percentage of cells positive for integrin α6 in the substantially pure population of pluripotent stem cell-derived airway basal-like cells is 92.4%.

The results in FIG. 1, graph 3A demonstrates that the percentage of control cells which are positive for NGFR in the control cell population is 2.65% and graph 3B demonstrates that the percentage of cells positive for NGFR in the substantially pure population of pluripotent stem cell-derived airway basal-like cells is 71.5%.

### Method for Obtaining an In Vitro Pluripotent Stem Cell-Derived Airway Epithelium Model

1. Obtain a substantially pure population of pluripotent stem cell-derived airway basal-like cells according to the method described above.
2. Coat inserts suitable for fitting to a well of a 24-well plate with a mix of Matrigel (1:20) and Fibronectin (1:100) diluted in phosphate-buffered saline (PBS) for at least 1 hour before use in an incubator at 37° C., 5% CO₂.
3. When pluripotent stem cell-derived airway basal-like cells reach confluency, add trypsin for 1 minute, aspirate trypsin, add fresh trypsin for 4 minutes then add stop medium. Centrifuge the cells at 400×g for 5 minutes, remove the supernatant and resuspended in fresh basal cell medium. Remove the inserts from the incubator and aspirate the PBS. Count the cells and dilute approximately 150,000 cells in 50 μl basal cell medium, plate on top of the insert in a 24-well plate. Add an additional 420 μl of basal cell medium to the bottom side of the insert. Incubate at 37° C., 5% CO₂.
4. After around 4 days, when the cells reach confluency, aspirate the medium from both sides of the insert. Add 420 μl PneumaCult medium (Stem Cell Technologies) to the bottom of the insert. Incubate at 37° C., 5% CO₂. The PneumaCult medium is replaced every 2 to 3 days on the bottom of the insert for around 3 weeks until mature.

### COVID-19 Model

The inventors have identified that the methods and resulting cells and lung models could be infected with coronavirus SARS-CoV-2 to give a COVID-19 lung model.

In summary, the inventors isolated a population of basal like cells from differentiating induced pluripotent stem cells and used these to generate airway epithelial equivalents by air-liquid interface (ALI) culture. They have shown that these comprise the cell types found in the human upper airway epithelium including functional ciliated cells, which are capable of secreting mucus and are readily infected by SARS-CoV-2 as demonstrated by the replication within the cells of the lung construct, release of virions into the supernatant growth media and the presence of SARS-CoV-2 spike protein in specific cells. Infected constructs also secrete cytokines.

### Generation of a In Vitro Pluripotent Stem Cell-Derived Airway Epithelium Model or Lung Model Using a Substantially Pure Population of Pluripotent Stem Cell-Derived Basal-Like Cells

Induced pluripotent stem cell lines SBAD2 and SBAD3 (StemBANCC) are cultured at 37° C.+5% CO2 on 6-well plates coated with Matrigel™ (BD, 354230) in mTeSRTM1 (StemCell Technologies) with daily media replacement. At 80% confluency, the cells are passaged with Versene EDTA 0.02% (Lonza) for 5 minutes and transferred at a split ratio of 1:3 into fresh matrigel-coated plates. The cells are passaged at least twice (and have approx. 80%-90% confluency) before initiating differentiation.

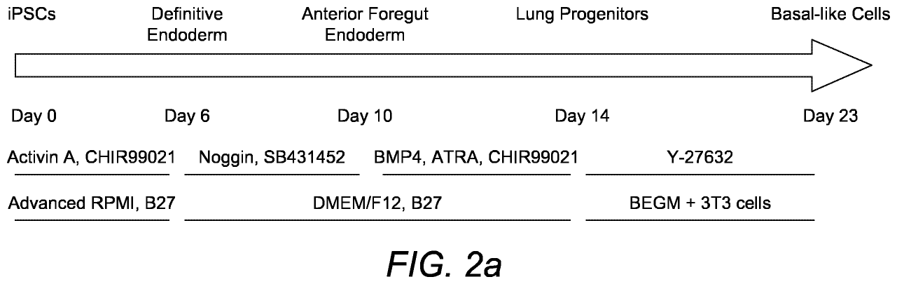

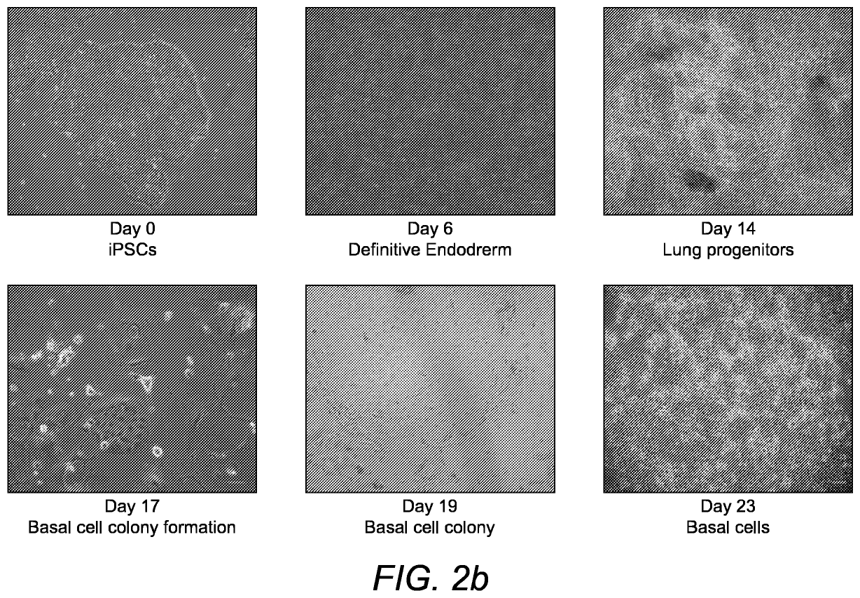

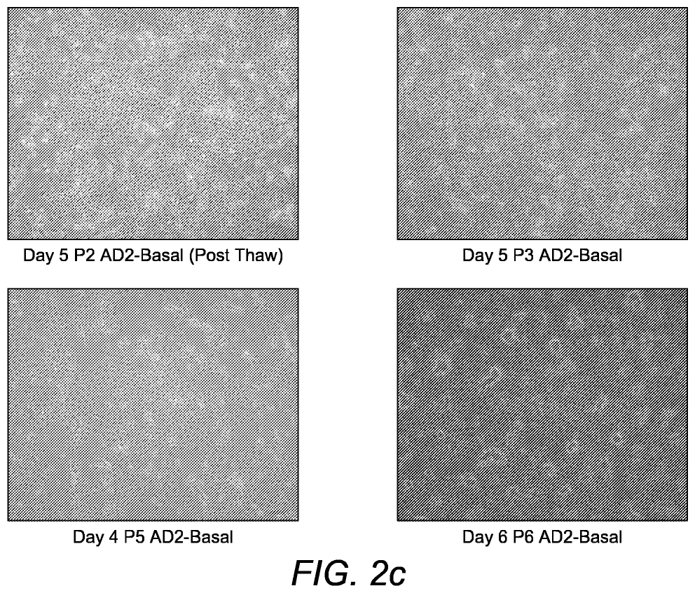

Differentiation into airway basal-like cells involves transit through definitive endoderm and anterior foregut endoderm stages as follows (FIG. 2a). Briefly, 90% confluent iPSCs are washed with PBS (2×2 ml per well of a six-well plate) then cultured in Advanced RPMI1640 medium (Gibco) containing 0.02% B27 supplement (Life Technologies) supplemented with 50 U/ml penicillin/streptomycin, 100 ng/ml human activin A (R&D systems), 1 μM CHIR99021 (sigma-aldrich), and 10 μM of Y-27632 (chemdea). The medium is refreshed daily for 6 days, and the cells are kept in an incubator at 37° C. containing 5% CO₂ and 95% humidity. On Day 6, the medium is changed to Advanced RPMI1640 medium (Gibco) containing 0.02% B27 supplement (Life Technologies) supplemented with 100 ng/ml human recombinant noggin (R&D systems) and 10 μM of SB-431542 (R&D systems). The medium is changed daily for 4 days. On Day 10, the medium is changed to Advanced RPMI1640 medium (Gibco) containing 0.02% B27 supplemented with 100 ng/ml of human recombinant BMP4 (Prepotech), 0.5 μM of all-trans retinoic acid (ATRA) (Sigma-Aldrich) and 3 μM of CHIR99021. The medium is changed every other day for 4 days. This provides a mixed population of lung progenitors, i.e. a population of pluripotent stem cell-derived airway basal like cells.

To isolate airway basal-like cells, i.e. to obtain a substantially pure population of pluripotent stem cell-derived airway basal-like cells, the day 14 differentiated cells are washed with PBS and enzymatically detached with trypsin for 5 minutes. The detached cells are centrifuged at 300×g and resuspended in BEGM medium (Lonza) supplemented with 10 μM of Y-27632. They are plated at low density e.g. at a ratio of 1 well into 6 on mitotically inactivated 3T3 cells (mitotically inactivated mouse 3T3-J2 feeder cells). The medium is changed every other day, until 90% confluency of basal cells is reached. The basal cells are passaged at a ratio of 7000 cells/cm² on irradiated 3T3s for at least 8 passages. The cells can also be frozen at 1 million per vial in 50% BEGM 40% FBS and 10% DMSO and stored in liquid nitrogen for later use. Colonies of epithelial like cells grew within 10 days of plating (see FIGS. 2b and 2c) and these colonies were found to express markers that have previously been identified as lung basal cells markers i.e. keratin 14 (KRT14), Integrin alpha 6, NGFR and ΔNp63. The markers were detectable by immunohistochemistry and quantifiable by flow cytometry. This is a substantially pure population of pluripotent stem cell-derived basal-like cells.

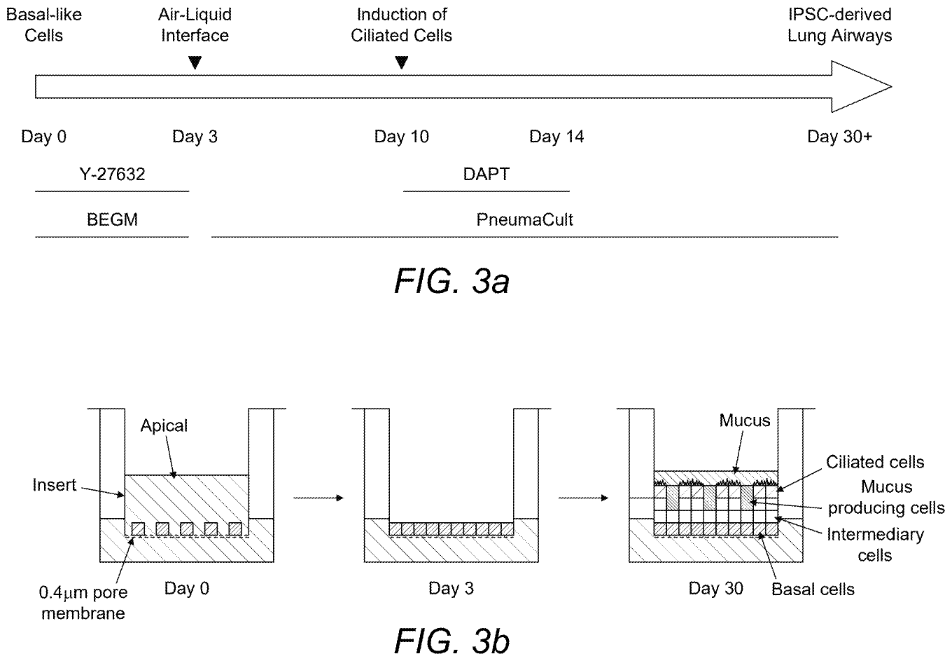

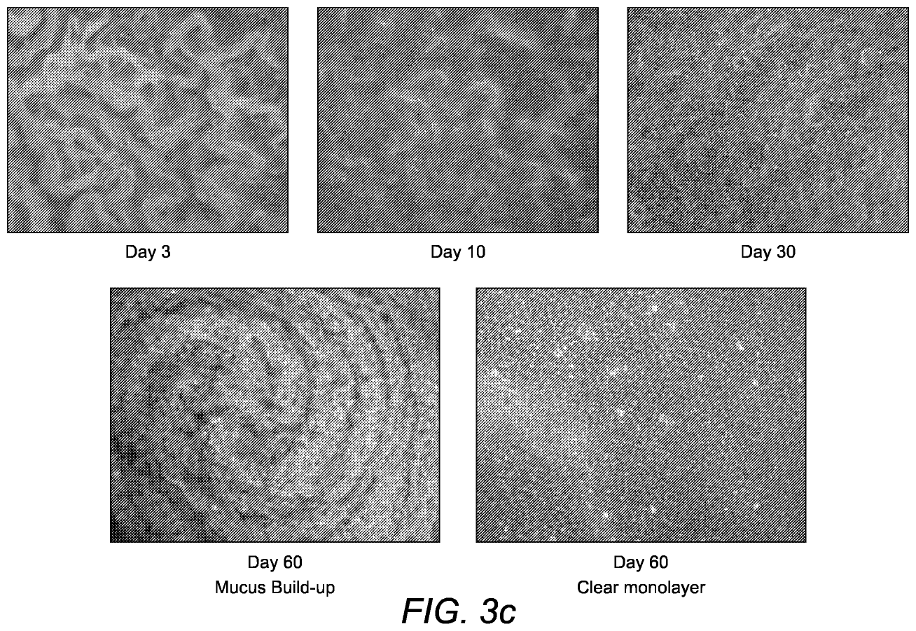

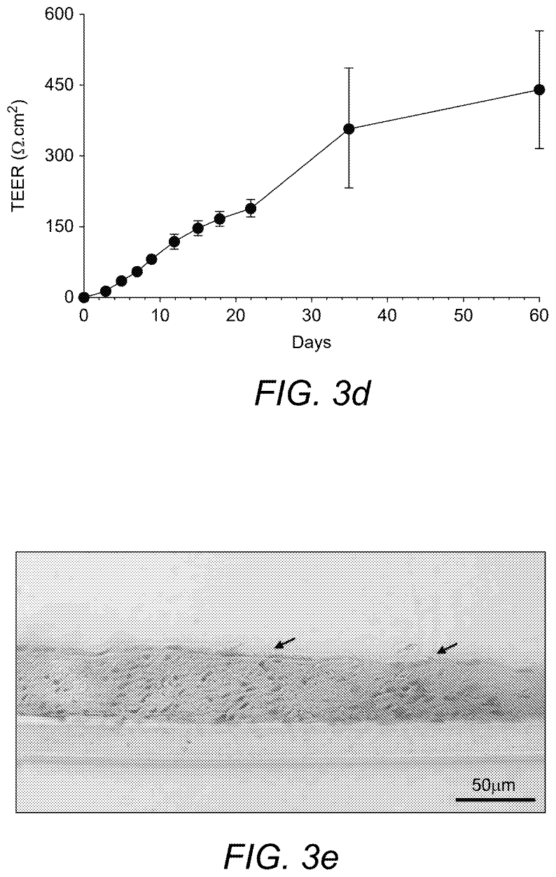

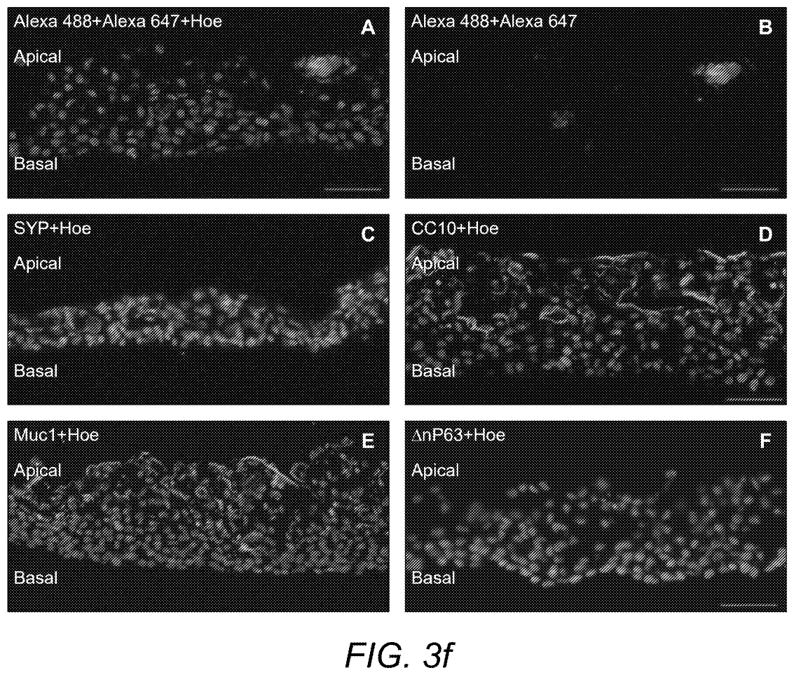

To then differentiate the airway basal-like cells into a pseudo-stratified airway epithelium, i.e. an in vitro pluripotent stem cell-derived airway epithelium model to act as a lung model (FIG. 3a), the obtained substantially pure population of pluripotent stem cell-derived airway basal-like cells, at 90% confluency on mitotically inactivated 3T3 feeders, are harvested as a single cell population by trypsinisation then seeded at a density of 150,000 cells per well onto the apical face of 24 well plate cell culture inserts (ThinCerts™, Greiner bio-one) with a transparent membrane (PET), with a pore diameter 0.4 μm, pre-coated with Matrigel (1:100) and fibronectin (1:100) (ThermoFisher) (FIG. 3b). The adherent cells are fed for three days apically and basolaterally with BEGM medium until they form a confluent monolayer. Once confluent, the apical medium is removed, and the cells are fed PneumaCult™ (Stem Cells Technologies) supplemented with heparin, hydrocortisone and Pen/Strep from the basal chamber. The cells are cultured for 4 weeks to achieve maturity and fed every other day from the basal chamber.

Cells with beating cilia were visible after 4 weeks of culture (FIG. 3c) and a mucus layer was present on the apical surface of the model with multiple hole like structures in which a concentrated amount of cilia could be observed (FIG. 3c, fourth panel). Trans epithelial electrical resistance (TEER) values increase to the range of 250-550 Ω/cm² by day 60 of culture indicating establishment of an epithelial barrier (FIG. 3d). Sections of the model indicate the presence of a pseudostratified epithelium (FIG. 3e) in which cells expressing the basal cell marker, p63 are enriched in a layer adjacent to the membrane of the transwell insert (FIG. 3f sixth panel). Differentiation of the basal cells into the other cell types present in the pseudostratified epithelium is indicated by the presence of club cell protein 10 (club cells, FIG. 3F, fourth panel), formation of tight junctions between ciliated epithelial cells (ZO-1 and acetylated tubulin) and expression of mucin-1 (goblet cells, FIG. 3F, fifth panel)

### Characterisation of Pluripotent Stem Cell-Derived Airway Basal-Like Cells and Pseudo-Stratified Airway Epithelium/Lung Model

Pluripotent stem cell-derived airway basal-like cells generated in the manner described above have been characterised by a combination of flow cytometry (Fortessa flow cytometer and FloJo analysis) and immunohistochemistry (IHC). At least 10,000 cells were analysed for each pluripotent stem cell-derived airway basal-like cell sample. For IHC analysis, pluripotent stem cell-derived airway basal-like cells were grown on feeder layers of mitotically inactive 3T3 cells on glass coverslips and cultured in 24 well plates. The 24-well plates containing coverslips were washed with PBS (2×1.0 ml) and fixed with 4% paraformaldehyde in PBS for 10 minutes at 37° C. The cells were washed with PBS (2×1.0 ml) then permeabilised with 1.0 ml of PBS-0.25% Triton X-100 for 30 minutes. The permeabilisation solution was replaced with blocking solution (2% BSA in PBS (w/v)) followed by incubation for 1 hour. Each primary antibody was diluted in 150 μL of the blocking solution at appropriate concentrations. The cells were then treated with the primary antibodies and incubated at 4° C. (12 hrs). Following this, the cells were washed with PBS (3×1.0 ml). Secondary antibodies were prepared in blocking solution and added on the samples for 1 hour at room temperature in the dark. The cells were washed again with PBS (3×1.0 ml). The coverslips were removed from the plate once the staining was finished and placed on a superfrost slide with a few drops of Vectashield medium containing Hoechst (as a nuclear counter-stain). Coverslips were sealed with nail polish and left to dry in a dark box before storage at 4° C. followed by fluorescence microscopy.

Mature air-liquid interface cultures prepared in the manner described above were fixed directly on the membrane with 4% paraformaldehyde for 10 minutes at 37° C. and then washed with PBS (3×1.0 ml). The tissues were then removed together with the membrane, placed into moulds, and covered with embedding matrix OCT (Cell Path). The moulds were placed at −20° C. to solidify. Once solid they were sectioned into 5 μm slices on slides using a cryostat. The sectioned membrane was removed with PBS washes and the slides were then stained using the same procedure as the basal cells using the same antibodies with the addition of antibodies against ACE2, TMPS2 and Synaptophysin. Once the staining was finalised, a few drops of Vectashield medium containing Hoechst were added to the slides then they were covered with long coverslips, sealed with nail polish, and left to dry at 4° C. Haematoxylin/Eosin stained sections were obtained as follows: ALI cultures were fixed by incubation with 4% PFA/PBS (w/v) for 10 minutes at room temperature. The membrane containing tissues were surgically removed from the insert and sandwiched between Shandon™ sponges (Thermo Scientific) and 3 mm whatman™ paper (GE Healthcare) in tissue embedding cassettes. Subsequently, paraffination was performed using the Excelsior™ AS Tissue processor (Thermo Scientific) and the paraffinated tissues were placed into moulds. Once solid, 3 μm sections were made using a microtome. The slides were rehydrated using xylene and an ethanol series (100%, 96% and 70%). Subsequently, the slides were stained using mayers hematoxylin (Sigma) and alcoholic eosin Y (Sigma) followed by dehydration using an ethanol series (80%, 96% and 100%). Xylene washed slides were mechanically covered using coverslips and dried at room temperature. Histology was assessed using an Axiovert 25 inverted microscope (Zeiss).

### Quantification of Cilia Beat Frequency

Prior to imaging, the apical surfaces of the ALI cultures, i.e. in vitro pluripotent stem cell-derived airway epithelium model, were washed using medium from the basal chamber. Subsequently, high-speed videos were captured using the Nikon Eclipse Ti2 LIPSI high content imaging microscope equipped with a ph1 phase contrast ring and a CFI S Plan Fluor LWD 20×objective. The middle of the Prime BSI sCMOS camera was used to capture 550 images (512×512 pixels) over 5.5 seconds at a rate of 100 frames per second. During imaging, the atmosphere was constantly kept at 37° C., 5% CO2 and 95% humidity. At least three fields containing cilia for three inserts were imaged.

For analysis MATLAB® was used to calculate cilia beat frequency. Here, the intensity-time trace of each pixel was filtered for frequencies between 5 and 25 Hz using a band pass filter. Subsequently, the power spectrum density was calculated. The frequency of each pixel is defined by the highest peak of the power spectrum density. The ciliated cells were visualised by plotting the frequency of each pixel into a heat map. The frequency distribution of the ciliated cells was visualized by plotting the amount of non-zero pixels into a histogram. The average frequency of all non-zero pixels was compared using a student's t-test.

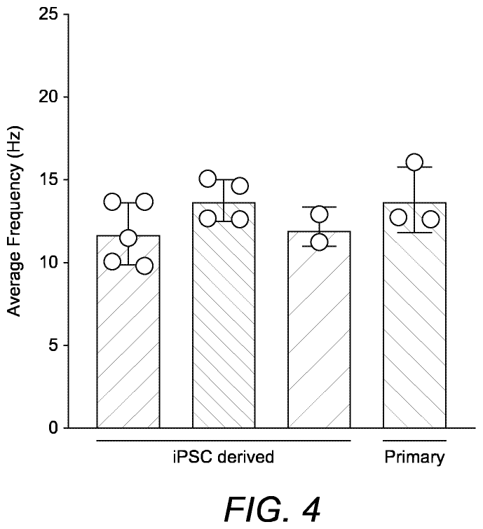

It was observed that the lung model, i.e. in vitro pluripotent stem cell-derived airway epithelium model, also referred to as iPSC derived basal cell ALI cultures, were comprised of patches of ciliated cells when compared to a more equally ciliated cell distribution for primary ALI cultures, however the beat frequency of cilia present on the iPSC derived models is in a similar range to those derived from primary basal cells (12.4±1.6 Hz and 13.6±2.0 Hz respectively—see FIG. 4).

### Infection of the In Vitro Pluripotent Stem Cell-Derived Airway Epithelium Model with SARS-CoV-2

To generate sufficient viral particles for infection experiments, SARS-CoV-2 isolate REMRQ0001/Human/2020/Liverpool was cultured from a clinical sample and passaged four times in Vero E6 cells (C1008; African green monkey kidney cells obtained from Public Health England) cultured in Dulbecco's minimal essential medium (DMEM) containing 10% fetal bovine serum (FBS) and 0.05 mg/mL gentamycin at 37° C. with 5% CO2. The fourth passage of virus was cultured in Vero E6 cells with DMEM containing 4% FBS and 0.05 mg/mL gentamycin at 37° C. with 5% CO2 and was harvested 48 hours post inoculation. Virus stocks were stored at −80° C.

Virus quantification was performed by standard plaque assays on Vero E6 cells plated at a density of 6×10⁵ cells per well of a six well plate. 100 μl aliquots of virus stocks over several dilutions were added to each well of a six well plate, covered with overlay medium then incubated at 37° C./5% CO₂ for 72 hours, fixed with 10% formalin and stained with 0.05% crystal violet solution. The number of non-coloured plaques counted in the crystal violet stained plate indicated the number of plaque forming units or PFU count of the viral dilution.

Based upon the PFU count of the virus stock, dilutions were prepared to infect the in vitro pluripotent stem cell-derived airway epithelium model or lung model that had been obtained as described above at multiplicities of infection (MOI) of 0.1 and 0.01 in DMEM with 4% FBS and gentamycin. 100 μl of virus dilution was added to the apical face of each airway epithelial model on cell culture inserts followed by addition of 250 μl of DMEM with 4% FBS and gentamycin. At set time points, supernatant media from the model was transferred to a 1.5 ml microcentrifuge tube then centrifuged (2500 rpm, 5 mins). 750 μl of supernatant was transferred to a 2 ml screw cap vial and stored at −80° C. until needed for viral quantification. Triton x-100 was added to the remaining supernatants to a final concentration of 0.5% followed by incubation (room temperature, 30 min) to inactivate SARS-CoV-2. The inactivated supernatants were stored in liquid nitrogen until needed for quantification of cytokines. The cellular component of the model at each time point was collected for quantification of viral particles present within cells and additional models for each timepoint were fixed with 4% paraformaldehyde for 30 min to provide infected models for analysis by IHC.

Quantification of viral particles present in the supernatant growth media and cellular mass was determined for each time point using the Vero E6 plaque assay method. IHC was also performed. The results were indicative of the in vitro pluripotent stem cell-derived airway epithelium model or lung model having been infected with SARS-CoV-2 to give COVID-19 lung model.

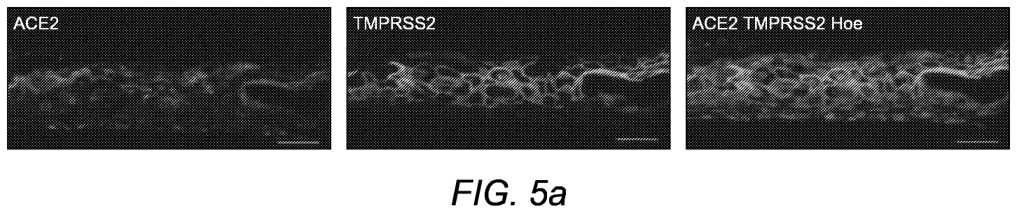

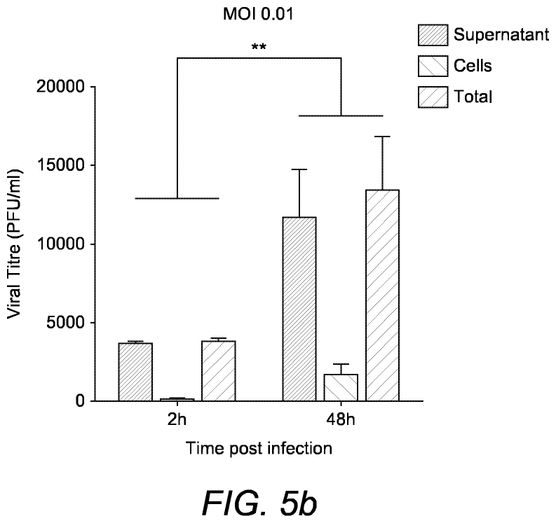

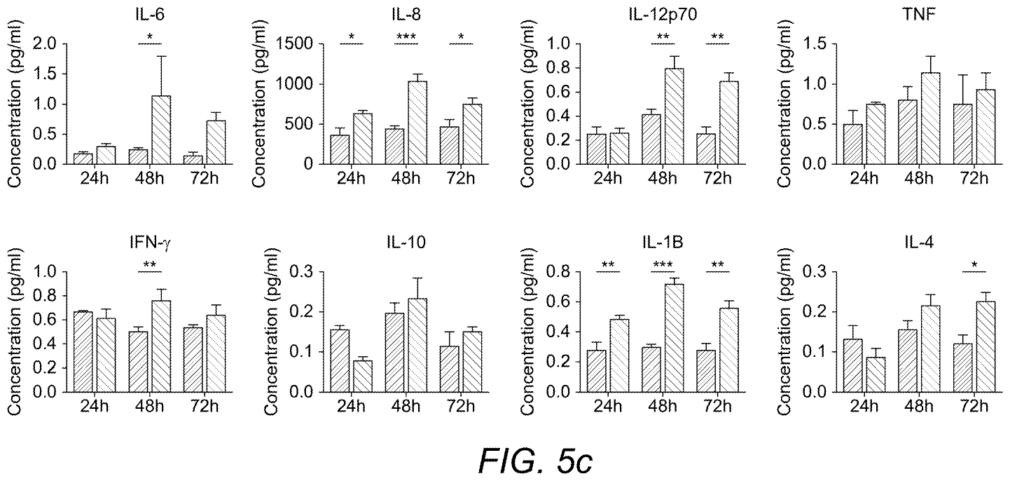

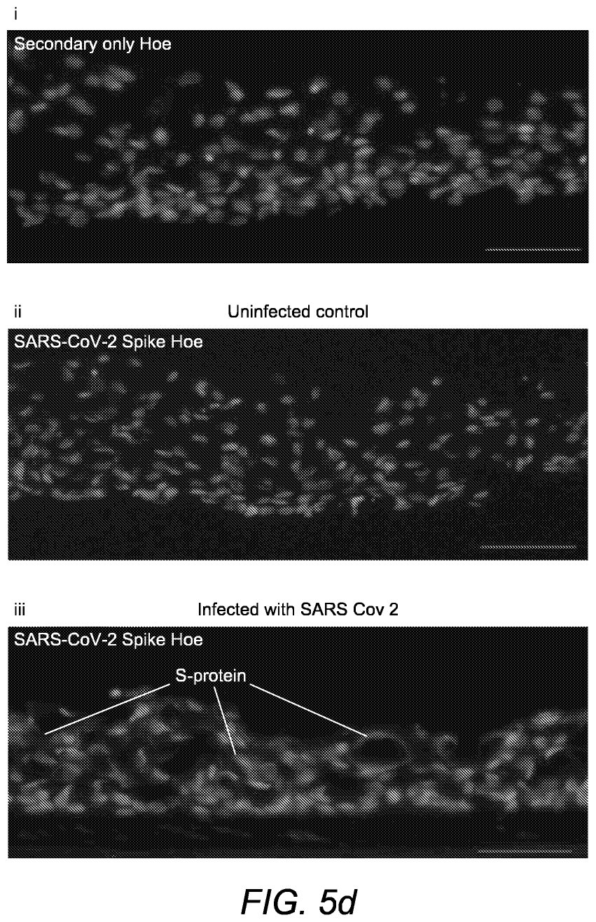

The SARS-CoV-2 genome encodes several structural proteins including the glycosylated spike protein (S-protein) that mediates cell invasion by binding to angiotensin converting enzyme 2 (ACE2) on the surface membrane of target cells. Cell invasion also requires S-protein priming which is facilitated by the host cell serine protease TMPRSS2. in vitro pluripotent stem cell-derived airway epithelium models or lung models generated according to the methods described above express both proteins required for SARS-CoV-2 invasion (FIG. 5a) and following inoculation of the virus onto the apical face of the model at an MOI of 0.01, the number of viral particles present in the cells of the model and in the supernatant growth medium increases by a factor of three 48 hours post infection (FIG. 5b).

in vitro pluripotent stem cell-derived airway epithelium models or lung models secrete inflammatory cytokines in response to SARS-CoV-2 infection (FIG. 5c). IL-6, IL-10 and TNFα all show increased secretion 48 hours after infection in line with expectation, however the greatest (and most statistically significant) increase is shown by IL-6 equating to a four-fold increase above that of the uninfected control 48 hours post infection. In patients with COVID-19, IL-6 levels are significantly elevated (>2.9 fold greater than non-diseased individuals) and this is associated with adverse clinical outcomes. The ability of the model to reflect this characteristic supports its similarity to in vivo tissue.

It was important to show if cells in the model had been infected by SARS-CoV-2 even if live virions can no longer be detected. To address this, the presence of S-protein was detected by immunohistochemical location (FIG. 5d, uninfected control model, secondary antibody only control and IHC localisation of S-protein) showing that infected cells are clearly visible in greater numbers of the apical face of the model. This correlates well with the position at which the viral inoculum was added to the model.

All of the above confirm that the COVID 19 lung model that can be obtained by the methods described herein, acts similarly to in vivo tissue, thus supporting its use as an ex-vivo (or in vitro) method of screening for prophylactic and therapeutic agents for COVID-19.

All of the features disclosed in this specification (including any accompanying claims, abstract and drawings), and/or all of the steps of any method or process so disclosed, may be combined in any combination, except combinations where at least some of such features and/or steps are mutually exclusive. Each feature disclosed in this specification (including any accompanying claims, abstract and drawings) may be replaced by alternative features serving the same, equivalent, or similar purpose, unless expressly stated otherwise. Thus, unless expressly stated otherwise, each feature disclosed is one example only of a generic series of equivalent or similar features. The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims are generally intended as “open” terms (e.g., the term “including” or “comprising” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations).

It will be appreciated that various embodiments of the present disclosure have been described herein for purposes of illustration, and that various modifications may be made without departing from the scope of the present disclosure. Accordingly, the various embodiments disclosed herein are not intended to be limiting, with the true scope being indicated by the following claims.

1. A method for obtaining a substantially pure population of pluripotent stem cell-derived airway basal-like cells comprising the steps:
differentiating a population of pluripotent stem cells to obtain a heterogeneous population of pluripotent stem cell-derived lung progenitor cells;
culturing the pluripotent stem cell-derived lung progenitor cells in the presence of feeder cells and a rho-kinase inhibitor to obtain a population of pluripotent stem cell-derived airway basal-like cells; and
culturing the pluripotent stem cell-derived lung progenitor cells and feeder cells in a serum-free medium to obtain a substantially pure population of pluripotent stem cell-derived airway basal-like cells.

2. The method of claim 1, wherein the population of pluripotent stem cells are induced pluripotent stem cells (iPSCs).

3. The method of claim 2, wherein the induced pluripotent stem cells are derived from a patient without any known genetic disorder or respiratory disease.

4. The method of claim 2, wherein the induced pluripotent stem cells are derived from a patient with a known genetic disorder or respiratory disease.

5. The method of claim 1, wherein the obtained substantially pure population of pluripotent stem-cell derived airway basal-like cells comprises cells expressing one or more airway basal cell markers selected from ΔNP63, NGFR, cytokeratin 14 and integrin α6, and wherein at least 70% of the obtained substantially pure population of pluripotent stem-cell derived airway basal-like cells express the one or more airway basal cell markers.

6. (canceled)

7. (canceled)

8. The method of claim 1, wherein the obtained substantially pure population of pluripotent stem-cell derived airway basal-like cells contains cells having a cuboidal morphology.

9. The method of claim 1, wherein the obtained substantially pure population of pluripotent stem-cell derived airway basal-like cells contains cells having enlarged nuclei.

10. The method of claim 1, wherein the obtained pluripotent stem cell-derived lung progenitor cells are plated at a 1:1 ratio with the feeder cells.

11. The method of claim 1, wherein the feeder cells are mouse fibroblast cells.

12. The method of claim 1, wherein the feeder cells are 3T3-J2 cells.

13. The method of claim 1, wherein the feeder cells are mitotically inactivated.

14. (canceled)

15. The method of claim 1, wherein the rho-kinase inhibitor is used at a concentration of between 5 μM and 30 μM.

16. A substantially pure population of induced pluripotent stem cell-derived airway basal-like cells obtained according to a method comprising:
differentiating a population of pluripotent stem cells to obtain a heterogeneous population of pluripotent stem cell-derived lung progenitor cells;
culturing the pluripotent stem cell-derived lung progenitor cells in the presence of feeder cells and a rho-kinase inhibitor to obtain a population of pluripotent stem cell-derived airway basal-like cells; and
culturing the pluripotent stem cell-derived lung progenitor cells and feeder cells in a serum-free medium to obtain a substantially pure population of pluripotent stem cell-derived airway basal-like cells, wherein at least 50% of the cells express NGFR and at least 70% of the cells express Integrin α6.

17. The substantially pure population of induced pluripotent stem cell-derived airway basal-like cells of claim 16 wherein the cells have a cuboidal morphology and/or enlarged nuclei.

18. A method of treating an individual having respiratory disease, comprising implanting a pluripotent stem-cell derived airway basal-like cells obtained by the method of claim 1.

19. (canceled)

20. The method of claim 1 further comprising culturing the population of pluripotent stem cell-derived airway basal-like cells on an air-liquid interface to obtain an in vitro pluripotent stem cell-derived airway epithelium model.

21. The method of claim 20, wherein the obtained in vitro pluripotent stem cell-derived airway epithelium model comprises cells expressing one or more airway epithelial cell markers selected from Club Cell Protein 10, Mucin 1, ΔNP63 and Acetylated Tubulin.

22. (canceled)

23. The method of claim 20, wherein the in vitro pluripotent stem cell-derived airway epithelium model has a substantially layered structure which resembles a naturally occurring airway epithelium and comprises a plurality of cell types selected from basal cells, ciliated cells, goblet cells and club cells.

24. The method of claim 20, wherein the air-liquid interface is provided by culturing the pluripotent stem cell-derived airway basal-like cells on an insert placed in a cell culture vessel.

25. The method of claim 20, wherein the air-liquid interface culture is allowed to mature for 5 or more days.

26. An in vitro pluripotent stem cell-derived airway epithelium or lung model which expresses one or more airway epithelial cell markers selected from Club Cell Protein 10, Mucin 1, ΔNP63 and Acetylated Tubulin; and which has a substantially layered structure resembling a naturally occurring airway epithelium and comprises a plurality of cell types selected from basal cells, ciliated cells, goblet cells and club cells.

27. (canceled)

28. The method of claim 20, comprising infecting the in vitro pluripotent stem cell-derived airway epithelium model with coronavirus SARS-CoV-2.

29. The in vitro pluripotent stem cell-derived airway epithelium or lung model of claim 26, which has been infected with coronavirus SARS-CoV-2.

30. The in vitro pluripotent stem cell-derived airway epithelium or lung model of claim 26 which comprises cilia having a cilia beat frequency of 11±1 Hz to 14±1 Hz.

31. The COVID-19 lung model of claim 29 wherein the cells show increased secretion of IL-6 after infection with coronavirus SARS-Cov-2 compared to a model where cells are not infected with coronavirus SARS-CoV-2.

32. The COVID-19 lung model of claim 31 wherein IL-6 secretion equating to greater than 2-fold increase above that of the uninfected control is seen within 48 hours post infection.

33. (canceled)

34. The method of claim 20, comprising:
infecting the in vitro pluripotent stem cell-derived airway epithelium model with coronavirus SARS-CoV-2 to give an infected model;
bringing a test agent into contact with the infected model; and
detecting or measuring a response in the infected model.
